(19) United States
(12) Patent Application Publication
(10) Pub. No.: US 2018/0028520 A1
(43) Pub. Date: Feb. 1, 2018

(54) METHODS AND PHARMACEUTICAL COMPOSITIONS FOR TREATMENT OF AMYOTROPHIC LATERAL SCLEROSIS

(71) Applicant: THE TRUSTEES OF COLUMBIA UNIVERSITY IN THE CITY OF NEW YORK, New York, NY (US)

(72) Inventors: Christopher Henderson, New York, NY (US); Hynek Wichterle, New York, NY (US); Derek Oakley, Brookline, MA (US)

(21) Appl. No.: 15/515,679

(22) PCT Filed: Sep. 29, 2015
(86) PCT No.: PCT/US15/53007

§ 371 Date: Mar. 30, 2017

Related U.S. Application Data
(60) Provisional application No. 62/057,509, filed on Sep. 30, 2014.

(51) Int. Cl. A61K 31/454

(52) U.S. Cl. CPC A61K 31/454

(57) ABSTRACT

The present disclosure provides, inter alia, methods and pharmaceutical compositions for treating or ameliorating amyotrophic lateral sclerosis (ALS). Among the various aspects of the present disclosure is the provision of methods and pharmaceutical compositions for treating or ameliorating ALS. Briefly, therefore, the present disclosure is directed to a method for treating or ameliorating an effect of amyotrophic lateral sclerosis (ALS) comprising administering to a subject in need thereof a modulator of a gene selected from the group consisting of Phospholipase D1 (PLD1).

### CROSS-REFERENCE TO RELATED APPLICATION

This patent application claims the benefit of U.S. Provisional Patent Application No. 62/057,509, filed Sep. 30, 2014, which is incorporated by reference herein in its entirety.

### FIELD OF THE DISCLOSURE

The present disclosure provides, inter alia, methods and pharmaceutical compositions for treating or ameliorating amyotrophic lateral sclerosis (ALS).

### BACKGROUND

Amyotrophic lateral sclerosis (ALS), also known as Lou Gehrig's Disease, is a motor neuron disorder that affects muscle control, leading to muscle spasticity, weakness, speaking and breathing disease. ALS patients typically die prematurely of respiratory failure within five years of diagnosis. Unlike other motor neuron diseases, such as spinal muscular atrophy (SMA), no single therapeutic cause has been identified and a cure has eluded researchers. Therefore, there are few drugs in the clinical research pipeline for ALS.

Far greater “therapeutic success” with ALS has been achieved by nature itself, both in the way certain motor neuron (MN) subpopulations are completely preserved until endstage, and through potent genetic disease modifiers. An SOD1 mutation that leads to onset in one family member at 18 years can be delayed until 72 years in another member of the same pedigree. In a recent report (Kaplan et al., 2014), by studying the molecular basis of the ALS resistance of oculomotor neurons, MMP-9 was discovered to be a candidate disease-modifying therapeutic target. Here the focus is on candidate disease modifiers deduced from age of onset in human patients, and whether they may play similar roles in other neurodegenerative diseases.

Accordingly, there is a need for novel candidate therapeutic targets for the treatment (including prevention) of ALS.

### SUMMARY OF THE DISCLOSURE

Among the various aspects of the present disclosure is the provision of methods and pharmaceutical compositions for treating or ameliorating ALS.

Briefly, therefore, the present disclosure is directed to a method for treating or ameliorating an effect of amyotrophic lateral sclerosis (ALS) comprising administering to a subject in need thereof a modulator of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPO; zinc finger RNA-binding motif sennetarginine rich 2 U2AF35-related protein (ZRSR2), and combinations thereof, in an amount effective to treat or ameliorate an effect of amyotrophic lateral sclerosis (ALS).

Another aspect of the disclosure is directed to a method for preventing or slowing motor neuron disease in a subject comprising administering to a subject in need thereof a modulator of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPO; zinc finger RNA-binding motif sennetarginine rich 2 U2AF35-related protein (ZRSR2), and combinations thereof in an amount effective to prevent or slow motor neuron disease.

Another aspect of the disclosure is directed to a pharmaceutical composition for treating or ameliorating an effect of amyotrophic lateral sclerosis (ALS) in a subject in need thereof, the pharmaceutical composition comprising a pharmaceutically acceptable carrier or diluent and an amount of a modulator of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPO; zinc finger RNA-binding motif sennetarginine rich 2 U2AF35-related protein (ZRSR2), and combinations thereof, which amount is effective to treat or ameliorate an effect of amyotrophic lateral sclerosis (ALS) in the subject.

Other objects and features will be in part apparent and in part pointed out hereinafter.

### BRIEF DESCRIPTION OF THE DRAWINGS

FIG. 1 depicts four examples of genes that show correlation with age of onset or ALS genotype. Post-hoc analysis of the gene expression data published by Rabin et al. (2010) reveals a series of genes whose expression level is correlated to age of disease onset. NBEAL1, PLD1, CASP3, and ZRSR2, which will be studied in more detail, are shown as examples.

FIG. 2 depicts altered Ca⁺⁺ handling in ALS motor neurons. Human iPSMNs were loaded with Fluo-4, then subjected to 10 iterative pulses of 100 μM KA spaced 2 min apart.

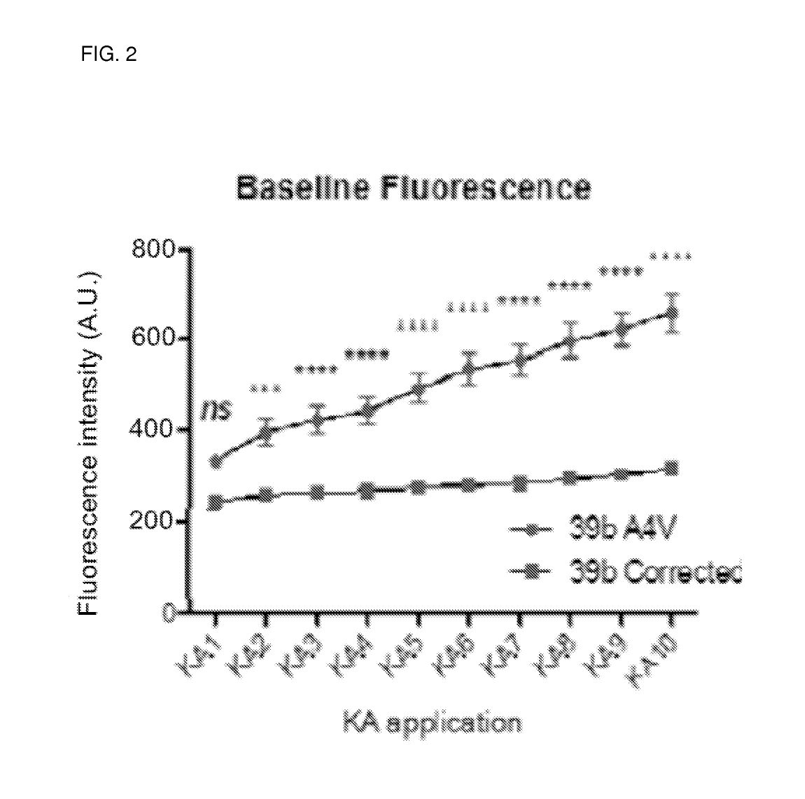

FIG. 3 depicts that ALS motorneurons (MNs) show increased spontaneous activity. Firing frequency judged by Ca⁺⁺ imaging in mouse ES-MNs.

FIG. 4 depicts an LC-MS analysis of phosphatidic acid species in the Pld1 KO forebrain. (A) PA species in Pld1 WT and KO forebrain. Values denote means+/−SE (n=6), *, P<0.05, **, P<0.01.

### DETAILED DESCRIPTION

The present disclosure is therefore based upon the reanalysis of data from post mortem gene expression in motor neurons of sporadic ALS patients. A series of genes were identified in which high expression levels are correlated with early disease onset. This expression pattern may reflect, for example, a role in increasing risk of ALS, or a homeostatic protective response in those motor neurons that survive the longest, thus determining whether one or more of those genes contribute either positively or negatively to disease onset.

By way of example, using a series of ALS-related phenotypes established in stem cell-derived motor neuron models, the effects of overexpression or knock-down of the gene(s) is examined. If overexpression accelerates degeneration, this will constitute evidence that the gene(s) is/are a risk factor, whereas if knock-down exacerbates the phenotype, the gene(s) is/are more likely to play a protective role.

ALS is driven by both cell-intrinsic and non-cell-autonomous factors but it is clear from the mutant SOD1 mouse model that the genetic status of MNs themselves plays a key role in determining disease onset (Boillée et al., 2006; Kaplan et al., 2014). Published microarray data from laser-captured MNs of ALS patients with widely varying age at death (Rabin et al., 2010) was analyzed. Although expression levels of the vast majority of genes were not linked to age, a subset of 43 genes showed remarkably strong correlation (R2>0.85). In all but one case, levels in captured MNs (but not surrounding tissue) were significantly higher in patients with earlier onset. If they play any functional role in ALS, these genes are potential negative disease modifiers. However, some of them may represent successful compensatory responses within those neurons that survive post mortem.

Either of these functions constitutes a potentially exciting novel therapeutic target with proof of concept in human, but to confer benefit a risk factor would need to be down-regulated and a compensatory response reinforced. Model systems provide one means to distinguish between these possibilities and we propose to use a combination of human iPS models in vitro and the SOD1G93A mouse in vivo to analyze the function of the most promising candidates.

Methods Related to ALS and Motor Neuron Disease

The present disclosure is therefore directed to methods for treating or ameliorating an effect of amyotrophic lateral sclerosis (ALS) in a subject. The present disclosure is also directed to methods for preventing or slowing motor neuron disease in a subject. The methods involve administering to a subject in need thereof a modulator of a gene related, directly or indirectly, to ALS.

The terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and pharmaceutical compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject, e.g., patient, population. Accordingly, a given subject or subject, e.g., patient, population may fail to respond or respond inadequately to treatment.

As used herein, the terms “ameliorate,” “ameliorating,” and grammatical variations thereof mean to decrease the severity of one or more symptoms of the particular condition or disease, e.g., ALS or motor neuron disease, in a subject.

A “subject” in accordance with this disclosure is typically a mammal, preferably, a human. In addition to humans, categories of mammals within the scope of the present disclosure include, for example, agricultural animals, domestic animals, laboratory animals, etc. Some examples of agricultural animals include cows, pigs, horses, goats, etc. Some examples of domestic animals include dogs, cats, etc. Some examples of laboratory animals include rats, mice, rabbits, guinea pigs, etc.

The term “gene” includes a nucleic acid sequence that when translated, transcribed, and otherwise processed (such as post-transcriptional or post-translational processing) results in a protein or polypeptide. The term “gene”, as used herein, also includes gene products, such as transcribed mRNA of the gene and/or the resultant protein/polypeptide. It is further noted that certain genes may be alternatively spliced, thus producing different isoforms of the protein.

The term “modulator” means an agent that elicits an effect on gene expression or protein activity level. For example, in one aspect of this embodiment, the modulator is an inhibitor of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPI); zinc finger RNA-binding motif sennetarginine rich 2 U2AF35-related protein (ZRSR2), and combinations thereof. In one particular embodiment, the gene is selected from the group consisting of Phospholipase D1 (PLD1); Intraflegellar transport 57 homolog (IFT57) (HIPPI); ALS2CR16: neurobeachin-like 1 (NBEAL1); Mitofusin 1 (MFN1); Protein geranylgeranyltransferase type I, beta subunit (PGGT1B), and combinations thereof. In another particular embodiment, the gene is Phospholipase D1 (PLD1). As used herein, an “inhibitor” means an agent that reduces or suppresses gene expression, the amount of protein, or protein activity. In another aspect of this embodiment, the modulator is an activator of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPI); zinc finger RNA-binding motif sennetarginine rich 2 U2AF35-related protein (ZRSR2), and combinations thereof. As used herein, “activator” means any agent that increases gene expression, the amount of protein, or protein activity level. In one particular embodiment, the gene is selected from the group consisting of Phospholipase D1 (PLD1); Intraflegellar transport 57 homolog (IFT57) (HIPPO; ALS2CR16: neurobeachin-like 1 (NBEAL1); Mitofusin 1 (MFN1); Protein geranylgeranyltransferase type I, beta subunit (PGGT1B), and combinations thereof. In another particular embodiment, the gene is Phospholipase D1 (PLD1).

“Wild type” or “WT” refers to that version of a gene most commonly found in nature.

The term “gene therapy” refers to any procedure that uses nucleic acids to heal, cure, or otherwise improve a condition in a subject. In gene therapy, nucleic acids need to be delivered into specific cells. Delivery methods include viral and non-viral means, which are known in the art. E.g., Patil et al., AAPS J. 7(1): E61-E77 (2005); Gascón et al., Non-Viral Delivery Systems in Gene Therapy (2013); Somiari et al., Molecular Therapy, 2(3), 178-187 (2000); Herweijer, H., and J. A. Wolff, Gene therapy 10(6): 453-458 (2003); and Nayerossadat et al., Advanced biomedical research 1(2):1-11 (2012).

The terms “prevent”, “preventing” and grammatical variations thereof mean to keep, e.g., ALS or motor neuron disease, from occurring in a subject. As used herein, the terms “slow”, “slowing” and grammatical variations thereof mean to delay, e.g., the onset or progression of ALS or motor neuron disease.

An “effective amount” of a modulator disclosed herein is that amount of such modulator that is sufficient to achieve beneficial or desired results as described herein when administered to a subject or in vitro to motor neuron cells. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a modulator according to the disclosure will be that amount of the modulator, which is the lowest dose effective to produce the desired effect.

“Nucleic acid” or “oligonucleotide” or “polynucleotide” used herein mean at least two nucleotides covalently linked together.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

The nucleic acid may also be a RNA such as a mRNA, tRNA, antisense RNA (asRNA), short hairpin RNA (shRNA), short interfering RNA (siRNA), double-stranded RNA (dsRNA), transcriptional gene silencing RNA (ptgsRNA), Piwi-interacting RNA, pri-miRNA, pre-miRNA, micro-RNA (miRNA), or anti-miRNA the latter of which are described, e.g., in U.S. Pat. Nos. 7,642,348 and 7,825,229 and Published International Application Nos. WO 2005/116250 and WO 2006/126040. An asRNA is a single-stranded RNA molecule with a nucleotide sequence complementary to a sense strand RNA, i.e., messenger RNA. Antisense RNA may be introduced into a cell to inhibit translation of a complementary mRNA by base pairing to it and physically obstructing the translation machinery. siRNA gene-targeting may be carried out by transient siRNA transfer into cells, achieved by such classic methods as lipid-mediated transfection (such as encapsulation in liposome, complexing with cationic lipids, cholesterol, and/or condensing polymers, electroporation, or microinjection). siRNA gene-targeting may also be carried out by administration of siRNA conjugated with antibodies or siRNA complexed with a fusion protein comprising a cell-penetrating peptide conjugated to a double-stranded (ds) RNA-binding domain (DRBD) that binds to the siRNA (see, e.g., U.S. Pat. No. 8,273,867). An shRNA molecule has two sequence regions that are reversely complementary to one another and can form a double strand with one another in an intramolecular manner. shRNA gene-targeting may be carried out by using a vector introduced into cells, such as viral vectors (lentiviral vectors, adenoviral vectors, or adeno-associated viral vectors for example). The design and synthesis of siRNA and shRNA molecules are known in the art, and may be commercially purchased from, e.g., Gene Link, Inc., Invitrogen/Life Technologies, Thermo Fisher Scientific, and GE Healthcare/Dharmacon.

The nucleic acid may also be an aptamer, an intramer, or a spiegelmer. The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), disclosed in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13). The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610). The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those disclosed in U.S. Pat. Nos. 5,235,033 and 5,034,506. Nucleic acids containing one or more non-naturally occurring or modified nucleotides are also included within the definition of nucleic acid. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e., ribonucleotides, containing a non-naturally occurring nucleobase instead of a naturally occurring nucleobase such as uridines or cytidines modified at the 5-position, e.g., 5-(2-amino)propyl uridine, 5-bromo uridine; adenosines and guanosines modified at the 8-position, e.g., 8-bromo guanosine; deaza nucleotides, e.g., 7-deaza-adenosine; O- and N-alkylated nucleotides, e.g., N6-methyl adenosine are suitable. The 2′-OH-group may be replaced by a group selected from —H, —OR, —R, -halo, —SR, —NH₂, —NHR, —NR₂, or CN wherein R is C₁-C₆ alkyl, alkenyl or alkynyl and halo is F, Cl, Br or I. Modified nucleotides also include nucleotides conjugated with cholesterol through, e.g., a hydroxyprolinol linkage as disclosed in Krutzfeldt et al., Nature (Oct. 30, 2005), Soutschek et al., Nature 432:173-178 (2004), and U.S. Pat. No. 7,745,608. Modified nucleotides and nucleic acids may also include locked nucleic acids (LNA), as disclosed in U.S. Pat. No. 6,316,198. Additional modified nucleotides and nucleic acids are disclosed in U.S. Pat. No. 8,114,985. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

The terms “peptide,” “polypeptide,” and “protein” are used interchangeably herein. In the present invention, these terms mean a linked sequence of amino acids, which may be natural, synthetic, or a modification, or combination of natural and synthetic. The term includes antibodies, antibody mimetics, domain antibodies, lipocalins, targeted proteases, and polypeptide mimetics. The term also includes vaccines containing a peptide or peptide fragment intended to raise antibodies against the peptide or peptide fragment.

The phrase “small molecule” includes any chemical or other moiety, other than polysaccharides, polypeptides, and nucleic acids, that can act to affect biological processes. Small molecules can include any number of therapeutic agents presently known and used, or can be synthesized in a library of such molecules for the purpose of screening for biological function(s). Small molecules are distinguished from macromolecules by size. In various embodiments, for example, the small molecules may have a molecular weight less than about 5,000 daltons (Da), less than about 2,500 Da, less than 1,000 Da, or less than about 500 Da. As used herein, preferably, the small molecule is an organic compound, which refers to any carbon-based compound other than biologics such as nucleic acids, polypeptides, and polysaccharides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity. In one preferred embodiment, the modulator is a small molecule.

Pharmaceutical Compositions

The present disclosure is also directed to a pharmaceutical composition for treating or ameliorating an effect of amyotrophic lateral sclerosis (ALS) in a subject in need thereof. The pharmaceutical composition comprise a pharmaceutically acceptable carrier or diluent and an amount of a modulator of one or more genes described herein. In one embodiment, the modulator is an activator of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPO; zinc finger RNA-binding motif sennetarginine rich 2 U2AF35-related protein (ZRSR2), and combinations thereof. In another embodiment, the modulator is an inhibitor of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPI); zinc finger RNA-binding motif sennetarginine rich 2 U2AF35-related protein (ZRSR2), and combinations thereof. In one particular embodiment, the modulator is an activator or inhibitor of a gene selected from the group consisting of Phospholipase D1 (PLD1); Intraflegellar transport 57 homolog (IFT57) (HIPPI); ALS2CR16: neurobeachin-like 1 (NBEAL1); Mitofusin 1 (MFN1); Protein geranylgeranyltransferase type I, beta subunit (PGGT1B), and combinations thereof. In one particular embodiment, the modulator is an activator or inhibitor of Phospholipase D1 (PLD1). In various embodiments, the modulator is a small molecule.

A pharmaceutical composition of the present disclosure may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a pharmaceutical composition of the present invention may be administered in conjunction with other treatments. A pharmaceutical composition of the present invention maybe encapsulated or otherwise protected against gastric or other secretions, if desired.

The pharmaceutical compositions of the invention comprise one or more active ingredients in admixture with one or more pharmaceutically acceptable carriers or diluents and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers or diluents are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier or diluent used in a pharmaceutical composition of the invention must be “acceptable” in the sense of being compatible with the other ingredients of the formulation and not injurious to the subject. Carriers or diluents suitable for a selected dosage form and intended route of administration are well known in the art, and acceptable carriers or diluents for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers or diluents and, optionally, one or more fillers, extenders, binders, humectants, disintegrating agents, solution retarding agents, absorption accelerators, wetting agents, absorbents, lubricants, and/or coloring agents. Solid pharmaceutical compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These pharmaceutical compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions of the present invention for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions of the present invention which are suitable for vaginal administration also include pessaries, tampons, creams, gels, pastes, foams or spray formulations containing such pharmaceutically-acceptable carriers or diluents as are known in the art to be appropriate.

Dosage forms for topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier or diluent. The ointments, pastes, creams and gels may contain excipients. Powders and sprays may contain excipients and propellants.

Pharmaceutical compositions of the present invention suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsulated matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above. Kits containing one or more doses of the pharmaceutical compositions of the present invention alone or as part of a combination therapy are also within the scope of the present invention.

The definitions and methods are provided herein to better define the present disclosure and to guide those of ordinary skill in the art in the practice of the present invention. Unless otherwise noted, terms are to be understood according to conventional usage by those of ordinary skill in the relevant art. The definitions used herein are for the purpose of describing particular embodiments only and are not intended to be limiting. As used herein, the singular forms “a,” “an,” and “the” include plural referents unless the context clearly dictates otherwise.

Having described the invention in detail, it will be apparent that modifications and variations are possible without departing the scope of the invention defined in the appended claims. Furthermore, it should be appreciated that all examples in the present disclosure are provided as non-limiting examples.

### Examples

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

This disclosure is innovative at technical, scientific and conceptual levels:

Novel approach: the first study in which candidate disease modifiers deduced directly from analysis of patient motor neurons will be functionally evaluated in human motor neurons in vitro.

Novel tools: isogenic control pairs of human iPS lines allow for unambiguous definition of cellular phenotypes linked to disease

Novel outcomes: unpublished ALS phenotypes linked to neuronal excitability model disease stages close to clinical onset, the phase most clearly affected by candidate modifiers.

Novel target: first study of the role of PLD1 and its product PA in ALS, based on proof-of-concept data from human patients.

Preliminary Data and Experimental Procedures

Identification of Candidate Disease Modifier Genes from Human Post-Mortem Studies.

Gene expression in MNs cannot be monitored in living patients, since a spinal cord biopsy would be highly invasive and lead to paralysis. Moreover, in adult spinal cord, MNs make up fewer than 2% of the total cells, so even post mortem gene expression analyses performed on spinal cord homogenates evaluate essentially only cells other than motor neurons. In 2010, Ravits and colleagues studied spinal cords from human ALS and control patients collected with a short post mortem delay (Rabin et al., 2010). They used laser capture microdissection to purify motor neurons from regions of the spinal cord that were relatively unaffected and then performed microarray gene expression analysis. This provides arguably the highest quality dataset for gene expression in ALS motor neurons.

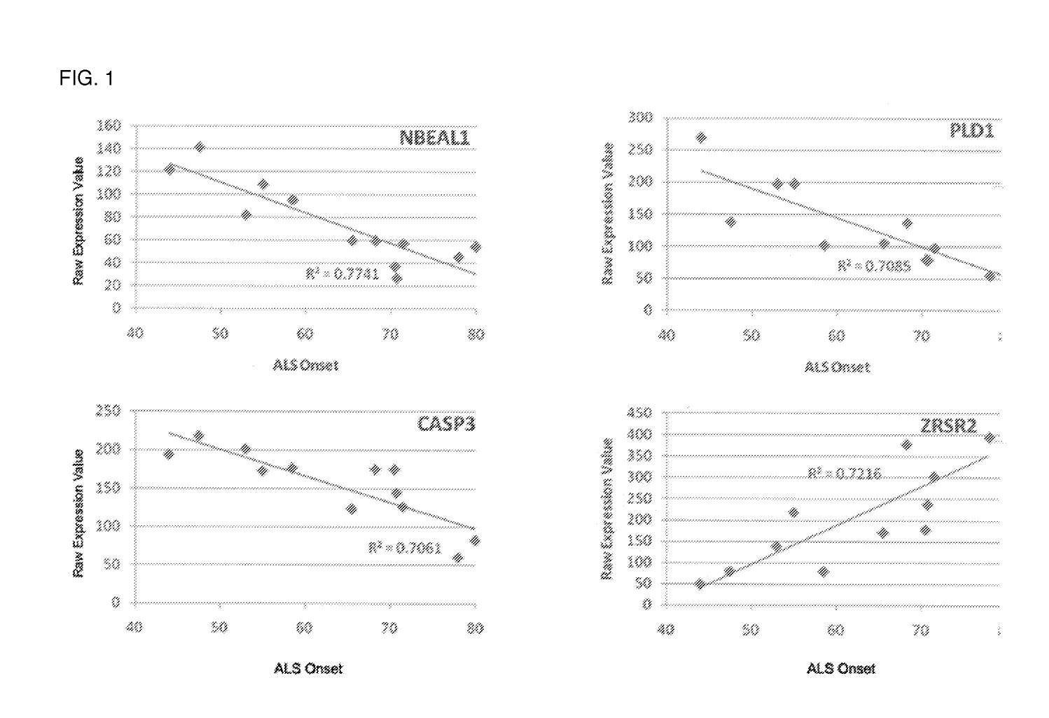

The authors of the study used the expression data to look for differences between ALS and control motor neurons. We instead reanalyzed the published dataset looking for genes whose expression varies with age of onset. Quantitative (QT) trait analysis was performed using “ALS age at onset” as the QT. Remarkably, 43 genes (Table 1) whose levels correlated with age at onset with P<0.001 were identified. FIG. 1 shows representative genes that correlate with age of onset or ALS genotype. Several arguments strongly suggest that the correlations were generated by chance. First, no genes were identified in MN samples from 10 healthy controls using age of death as the QT. Moreover, none of the genes identified in ALS patients were correlated with age of death in the control patients when tested one by one. Second, we asked whether random assignment of age-at-onset values within the ALS group would identify another set of apparently correlated genes but this produced very few onset-correlated genes, meaning that any artificial effect of multiple comparisons is small. Overall, therefore, our analysis identifies an entirely novel set of genes whose expression levels may affect the severity of the ALS disease process. However, it is also possible that some or all of them reflect responses to the disease process in those motor neurons that survive until the patient's demise. Whichever is true, since these genes are shared across a diverse population of 12 patients with the sporadic form of the disease—and therefore presumably diverse disease triggers and mechanisms—it is likely the modifiers have a general effect on motor neuron viability rather than intervening in a specific mechanism (e.g. changing levels of a disease gene).

TABLE 1

| Correlation | Parametric- | | |
|---|---|---|---|
| coefficient | valued | Gene symbol | Description |
| -0.916 | <1e−07 | PLD1 | phosphtpase D1, |
| | | | phosplantidylchlorine-specific |
| -0.979 | <1e−07 | AIMP1 | aminoacyltRNA synthetase |
| | | | complex-interacting |
| | | | multifuctional protein 1 |
| -0.937 | <1e−07 | SLC26A2 | solute carrier fumity 25 |
| | | | (sulfate transporter) member 2 |
| -0.93 | <1e−07 | PCRD3 | polymerase (DNA-directed), |
| | | | delta 3, accessory aubunit. |
| -0.909 | <1e−07 | PTPRM | protein tyrosine phosphatinse, |
| | | | receptor type, M |
| -0.895 | 5.90H-06 | SOAT1 | sterol O-acytransferase 1 |
| -0.888 | 9.17E-05 | HEL | v-rel reticulnendothellosis |
| | | | viral onengerate buntnolog |
| -0.888 | 9.17E-05 | C1GALT2 | core 1 synthases, glycoprotein- |
| | | | N-acetylgalectocamine |
| | | | 3-beta-galactosyltranferese |
| -0.888 | 9.17E-05 | NAA38 | LSMS: N(alphia)-acetyltransferase |
| | | | 38, NalC auxillary submit |
| -0.881 | 0.0001922 | PIBF1 | progesterone Inmumiontridity |
| | | | binding factor 1 |
| -0.874 | 0.0003089 | KDM5B | lysine (K)-specific demethylase 5B |
| -0.874 | 0.0003059 | MFN1 | multufusin 1 |
| -0.874 | 0.0003089 | MRPL1 | mltochondrial ribosonal protein L1 |
| -0.874 | 0.0003089 | MOP-1 | mRNA for MOP-1 |
| -0.874 | 0.0003089 | ARL4A | ADP-ribosythution factor-like 4A |
| -0.864 | 0.0003089 | ZRSR2 | zinc finger (CCCH type), RNA-binding |
| | | | motif and serine/arginine rich 2 |
| | | | (U2AF35-related protein) |
| -0.867 | 0.0004433 | SLC3CA7 | solute carrier family 30 |
| | | | (zine transporter), member 7 |
| -0.867 | 0.0004433 | ZNF67B | zinc finger protein 678 |
| -0.867 | 0.0004433 | RALB | v-ral simlon lethemin viral |
| | | | uncogene homolog B |
| -0.867 | 0.0004433 | PRMT10 | protein arglini methyltransferase 10 |
| -0.867 | 0.0004433 | SEMA3D | memaphorin 3D |
| -0.867 | 0.0004433 | C9orf102 | chrosome 9 open reading frame 102 |
| -0.867 | 0.0004433 | ARFGAP3 | ADP-ribosylation factor GTPase |
| | | | activating protein 3 |
| -0.86 | 0.0005971 | WDR3 | WD repeat domain 3 |
| -0.86 | 0.0005971 | EML4 | echinoderm microtuble associated protein like 4 |
| -0.86 | 0.0005971 | NEAL1 | ALS2CR16: neurobeachin-like 1 |
| -0.86 | 0.0005971 | DUSP11 | dual specificity phosphotase 11 |
| | | | (RNA/RNP cumplex 1-interacting) |
| -0.86 | 0.0005971 | MFSD1 | major faclitor superfunny domain containing 1 |
| -0.86 | 0.0005971 | SLC4A7 | solute carrier family 4, sodium |
| | | | bicarbonate contransporter, member |
| -0.853 | 0.0007719 | INPP1 | INPP1 |
| -0.853 | 0.0007719 | PGGT1B | PGGT18 |
| -0.853 | 0.0007719 | TAS2R4 | TAS2R4 |
| -0.853 | 0.0007719 | TNFRSF11B | TNFRSF118 |
| -0.853 | 0.0007719 | TGFBR1 | TGFBR1 |
| -0.853 | 0.0007719 | LOC100131642 | LOC100131642 |
| -0.853 | 0.0009695 | C1orf27 | C1orf27 |
| -0.846 | 0.0009695 | RAB7L1 | RAB7L1 |
| -0.846 | 0.0009695 | IFT57 | IFT57 |
| -0.846 | 0.0009695 | SRP72 | SRP72 |
| -0.846 | 0.0009695 | CASP3 | CASP3 |
| -0.846 | 0.0009695 | UTP15 | UTP15 |
| -0.846 | 0.0009695 | HIST1H1BC | HIST1H2BC |
| -.0846 | 0.0009695 | IMID1C | IMID1C |

Shortlist of Candidate Genes.

The number of candidate genes identified exceeds the number that can be reasonably studied. We have therefore prioritized them according to degree of differential expression and biological rationale and selected a group of 16 candidate genes (Table 2).

All of these have potential interest on the basis of their known activities but we propose initially to prioritize five of them:

Phospholipase D1 (PLD1): The gene to be studied with highest priority is PLD1, an enzyme responsible for the hydrolysis of phosphatidylcholine into phosphatidic acid (PA) and choline. Although PLD1 is involved in many cellular processes, including acetylcholine biosynthesis, two particularly ALS-relevant functions of its lipid product PA involve the regulation of neurite outgrowth and starvation-induced autophagy through activation of a variety of PA-binding protein effectors as well as direct physical effects on cellular membranes (Cai et al., 2006; Dall'Armi et al., 2013; Dall'Armi et al., 2010; Yoon et al., 2005). Additionally, we have shown that inhibition of PLDs with 5-fluoro-2-indolyl des-chlorohalopemide (FIPI) leads to increased insoluble tau aggregates in brain slices derived from a mouse model of tauopathy (Dall'Armi et al. 2010), whereas lack of PLD1 confers protection in a mouse model of AD by diminishing the amyloid burden (Point Du Jour et al., 2014).

HIPPI: High HIPPI expression shows a strong correlation (R=−0.85; p=0.001) with early-onset ALS in our analysis. HIPPI can form heterodimers with HIP1 (huntingtin interacting protein 1) that bind procaspase-8 (an intermediate in the motor neuron Fas pathway) thereby activating it (Gervais et al., 2002). HIPPI acts as a positive transcriptional regulator of caspases and REST (Datta and Bhattacharyya, 2011), and its expression triggers apoptosis in different neuroblastoma cell lines. Our preliminary data show this is also true in human iPS-MNs. We constructed a lentivirus expressing the full-length HIPPI cDNA and nIsGFP to identify infected neurons under the CMV promoter. FACS-sorted iPS-MNs were infected at 3 days post-plating and survival of infected motor neurons was counted at 7 days postinfection. Survival of infected iPS-MNs was significantly reduced as compared to uninfected cultures: 20±10% of control for iPS-MNs from healthy subjects and 23±12% for two ALS lines. These data provide proof of principle for the gain-of-function studies proposed and suggest that high levels of HIPPI can indeed contribute to motor neuron degeneration.

Neurobeachin-Like 1 (NBEAL1):

NBEAL1 is expressed broadly in neural tissue and contains a vacuolar targeting motif as well as PH-BEACH and WD40 domains (Chen et al., 2004). It is homologous to neurobeachin and ALFY. Neurobeachin (NBEA) is involved in neuronal membrane trafficking required for the development of functional neuromuscular junctions as well as synapses and dendritic spines in the CNS (Medrihan et al., 2009; Niesmann et al., 2011; Su et al., 2004; Wang et al., 2000). ALFY is a recently described regulator of autophagy involved in the selective targeting of protein aggregates to autophagosomes, mediating their autophagic clearance in the nervous system (Filimonenko et al., 2010). ALFY also binds p62, which has been implicated in both familial and sporadic forms of ALS (Fecto et al., 2011). We speculate that high levels of NBEAL may be a neuroprotective response.

Mitofusin 1 (MFN1):

MFN1 and the related protein mitofusin2 (MFN2) are GTPases critical for mitochondria fusion (Koshiba et al., 2004; Zorzano et al., 2010). The balance of mitochondrial fusion and fission, in turn, determines mitochondrial size and is important for an array of mitochondrial functions including trafficking, biogenesis, and overall health (Chen and Chan, 2005). Mitochondrial dysfunction is believed to contribute to ALS (Schon and Przedborski, 2011), consistent with a disease-modifying role for this enzyme. As described for HIPPI, our preliminary data indicate that overexpression in human iPS-MNs can trigger their degeneration. Our hypothesis is that mitofusin is a negative disease modifier.

Geranylgeranyltransferase 1 (GGT1):

GGT1 is one of the enzymes involved in protein prenylation, the addition of short lipid moieties to diverse proteins, such as RhoGTPases, to allow them to form signaling complexes at the plasma membrane. In unpublished studies with B. Stockwell and M. Filbin we screened 50,000 small molecules for their ability to enhance motor neuron axon growth. The most active were the cholesterol-lowering drugs statins, which were 500-fold more potent than the benchmarking compound Y27632, an inhibitor of Rho kinase. We subsequently showed that statins stimulate axon growth not by inhibiting cholesterol synthesis but by inhibiting protein prenylation. Accordingly, a combination of GGT and farnesyltransferase inhibitors completely overcame the inhibition of motor axon growth on cell lines expressing myelin-associated glycoprotein (MAG). We hypothesize that high levels of GGT may diminish the ability of motor axons to sprout, leading to reduced adaptive plasticity and earlier onset.

Gain- and Loss-of-Function Studies in SOD1G93A Mice In Vivo.

In Aim 1.2 and future studies we will use breeding to knockout strains and AAV viral vectors to modulate gene expression in ALS model mice in vivo. Our published data concerning MMP-9 (matrix metalloproteinase-9; Kaplan et al., 2014) illustrate the feasibility of these approaches and so they are described only briefly here. Our data suggested that MMP-9 might contribute actively to disease onset. We tested this in two ways. First, mmp9 knockout mice were crossed to the SOD1^{G93A} model. As our main endpoint, we focused on the earliest morphological change reported in these mice: denervation of fast muscle fibers. This endpoint defines the clinical onset of paralysis and occurs with the same specificity in human sALS patients. In the absence of MMP-9, we observed an 80-day delay in denervation, reflected in a >50% protection of compound muscle action potentials (CMAP) and motor coordination measured by rotarod. Median lifespan was increased by 39 days (or 25%). Therefore, we have robust and predictive assays for ALS disease modifiers.

For candidate genes for which there is no available knockout, we routinely use a single neonatal i.c.v. injection of AAV6, which leads to selective transduction of a 50-80% of all spinal motor neurons, but few other spinal cord cells. Using this approach AAV6-shRNA to Mmp-9 preserved muscle innervation to an extent comparable to that provided by heterozygote germline deletion. We recently worked with the viral vector core at UNC to optimize the serotype, titer and salt concentration of vectors for motor neuron transduction and all vectors will be ordered from this single source to optimize quality control.

ALS-Specific Survival Outcome In Vitro is Related to ER Stress.

Cultured mouse motor neurons with an ALS genotype do not show robust spontaneous survival deficits as compared to wildtype controls (Raoul et al., 2002). In order to reveal ALS-related differences in vulnerability we screened a collection of small molecules for agents that would induce death of SOD1^{G93A} ES-MNs but not control ES-MNs that overexpress wildtype human SOD1. The most selective compound was CPA, cyclopiazonic acid, which blocks the SERCA calcium pump in the endoplasmic reticulum and thereby triggers ER stress (not shown). CPA-treated SOD1^{G93A} ES-MNs therefore provide a system in which to test the ability of different agents to protect against ALS-specific cell death, and we have used this assay in unpublished experiments to identify neuroprotective small molecules. Here, we will use this ALS-specific assay to evaluate the effects of modulating levels of PLD1 and other candidate modifiers.

Effects of Mutant SOD1 Independent of Genetic Background: Isogenic Human ES/iPS Lines.

One of the obstacles of using patient-derived iPS cells is that a difference observed between an ALS iPS line and a control may not be related to the ALS genotype, but instead to other differences in the genetic background. Although this may be partly circumvented by the use of multiple ALS and control samples, the extreme inter-individual diversity in the human population means that very large numbers are required. We have therefore adopted the strategy of making gene-corrected derivatives of iPS lines bearing known point mutations. Thus, we recently took one of the SOD1A4v lines (#007) derived through an NIH GO grant and used Zn fingers to correct the mutation, forming an isogenic control line in which both SOD1 alleles have the wild-type sequence. The Eggan laboratory has shared a similar modification of another SOD1A4v line (#39b) we derived in collaboration. Lastly, we have introduced the A4V mutation into the SOD1 gene of the widely used HBG1 hESC line, which expresses GFP under the control of the motor neuron-specific HB9 promoter. We have used these isogenic pairs to directly demonstrate the relevance to ALS of a series of assays (below) that will be used to assess the effects of PLD1 and other candidate modifiers.

Altered Ca⁺⁺ Handling in ALS hiPS-MNs.

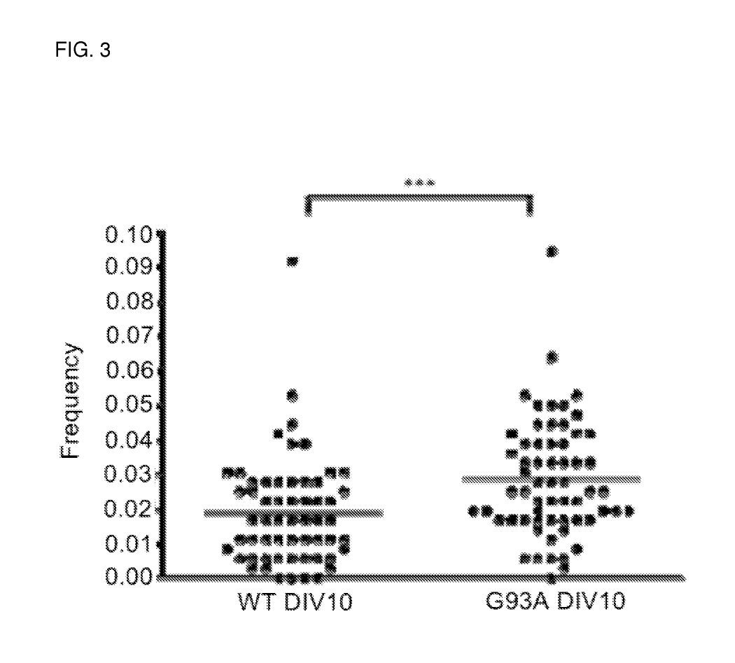

The effects of SERCA inhibition, and an extensive literature on hyperexcitability of ALS motor neurons at early stages of the disease suggest that ALS motor neurons may have intrinsic defects in responses to glutamate or Ca⁺⁺ handling by intracellular stores. To assess whether there are indeed functional differences between ALS MNs and controls at this level, we studied Ca⁺⁺ dynamics following multiple sequential applications of kainic acid (KA). In this assay, MNs are first treated with Fluo-4, a dye that binds free calcium within the cell, and then rapidly bathed in KA ten times at 2-minute intervals. The magnitude of KA-induced calcium transients can then be calculated for each cell. Using the 39b SOD1-A4V line and the isogenic gene-corrected control (see above), we found that ALS MNs are more excitable by kainate than control MNs, and also recover less completely (FIG. 3). This provides an ALS-relevant outcome measure for studying the role of candidate disease onset modifiers.

Excitability-Related Changes in Mouse and Human ALS ES/iPS-MNs.

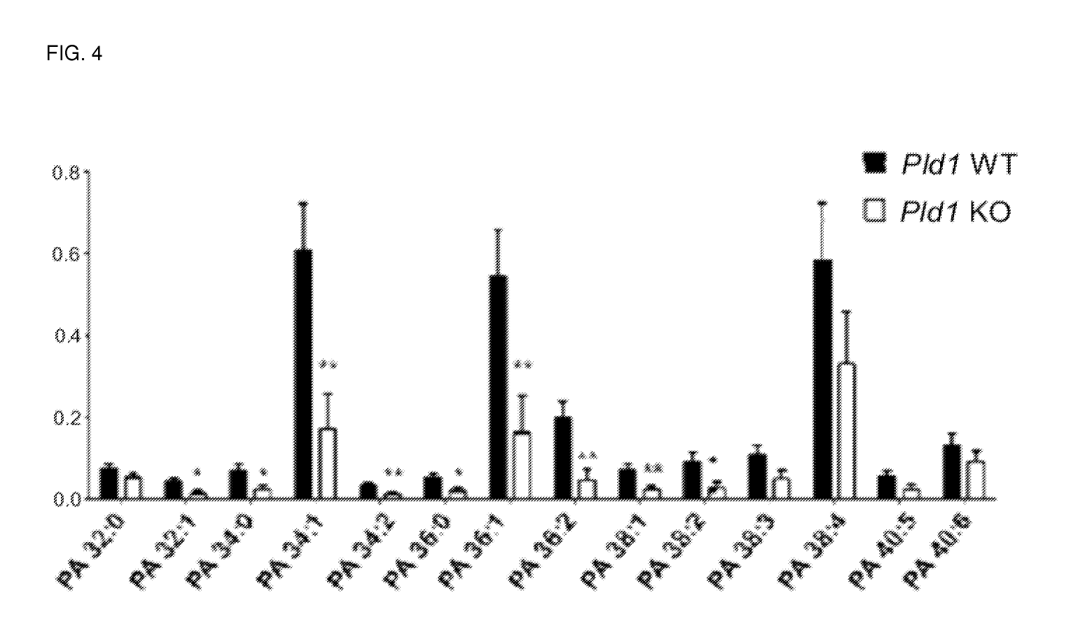

Using multielectrode arrays, Wainger et al. (2014) reported increased frequency of spontaneous action potential firing in ALS vs. control motor neurons. To analyze mouse ES-MNs from mice expressing SOD1G93A or SOD1wT we used a combination of Ca++ imaging and loose patch recording. ALS mES-MNs too showed increased rates of firing of action potentials and synaptic activity (p<0.001; FIG. 4). We looked for morphological correlates of this hyperexcitability that might also serve as endpoints. We found that both mouse and human ALS MNs show a significant shortening of the AIS (axon initial segment), as detected by immunostaining for ankyrin G (not shown). The AIS is the origin of action potentials and a reduction in AIS length would be expected to lead to reduced spontaneous activity (Kuba et al., 2010). Since that is not observed, it is likely that the reduction of AIS length reflects an attempt by the cell to compensate for hyperexcitability. In support of this, the shortening of the AIS in ALS iPS-MNs is further exacerbated by kainate and attenuated by TTx blockade (not shown).

Non-Cell-Autonomous Influences in ALS: Humanized In Vitro Model of Sporadic and Familial Forms.

The goal of the present invention is to identify disease modifiers that will protect motor neurons against multiple non-cell autonomous triggers of disease. One example is the well-demonstrated toxic effect of mouse and human ALS astrocytes (Di Giorgio et al., 2008; Haidet-Phillips et al., 2011; Nagai et al., 2007; Yamanaka et al., 2008). In collaboration with the Przedborski group, we recently devised a humanized co-culture model composed of human adult primary ALS astrocytes from fresh post mortem samples and human ES-MNs (Re et al., 2014). Death of MNs triggered by either sALS or fALS astrocytes in this system occurs through necroptosis. This provides a highly ALS-relevant assay to assess modifiers.

Specific Aim 1: PLD1 as a Candidate Disease Modifier in ALS.

High levels of PLD1 would be predicted by our data to exacerbate the ALS phenotype, as they do in AD mice. However, data from the Di Paolo lab show that PLD1 is also involved in triggering autophagy, which might be beneficial given the role of protein misfolding in ALS (Saxena and Caroni, 2011). It is important to distinguish between these potentially opposing effects in order to evaluate PLD1 as a candidate target. We will therefore assess the effects of modulating PLD1 activity on different ALS-related outcomes in vitro and in vivo.

Aim 1.1. Effects of PLD1 Modulation on Motor Neuron Degeneration In Vitro.

To assess the effects of increasing and decreasing PLD1 levels we will use the full range of ALS-related outcomes described above for mouse and human ES/iPS-MNs. These will include not only lines from familial SOD1 patients and their isogenic controls but also other lines from sporadic patients (3 lines) and other familial mutations (C9ORF72, angiogenin, TDP43 and FUS) that are already growing in the laboratory. This will be performed using both lentiviral shRNA/cDNA vectors, which infect iPS-MNs with −100% efficiency, and commercially available small-molecule PLD1 inhibitors (Selvy et al., 2011). We already have a validated lentiviral cDNA preparation and are currently screening the Sigma shRNA library available to us for the best shRNA. The small-molecule inhibitors have been successfully used in vitro in our AD studies.

Aim 1.2. Genetic Evaluation In Vivo of the Role of PLD1.

Pld1 KO mice have no apparent motor phenotype (Dall'Armi et al., 2010). Breeding of SOD1^{G93A} mice to the PLD1 KO strain on a homogeneous C57BL/6 background is nearly complete and should generate compound mutants before the start of the funding period. Mice that are homozygous, heterozygous or null for Pld1 will be evaluated using the criteria described above. Power calculations have been performed to determine the optimal group size for each outcome. Sufficient data to quantify muscle denervation are obtained from 5 mice only 2 months after birth. As a pharmacodynamic marker for these studies (and future experiments using small-molecule inhibitors in vivo), we will monitor PA levels in the spinal cord of mutant animals. The Di Paolo lab directs the lipidomic core of the Department of Pathology and Cell Biology and has set up standard operating procedures for lipid extraction and lipidomic analyses. We use a state-of-the-art Agilent 6490 Triple Quadrupole mass spectrometer (MS) combined with an Agilent 1290 liquid chromatography (LC) system for the operation of this LC/MS/MS platform, allowing us to cover four families of lipids (sterols, glycerophospholipids, glycerolipids and sphingolipids), and about thirty lipid subclasses for a total of 300-350 individual lipid species. Among these lipids, particular attention will be given to the various molecular species of PA (FIG. 5), as well as PA metabolites, such as DAG. Expected outcome and potential pitfalls. This aim should provide evidence for involvement of PLD1 in models of ALS. The work should be feasible within Year 1 of the grant since (a) tools for PLD1 modulation are available in the Di Paolo lab; (b) all the motor neuron assays in vitro are run routinely in the Henderson and Wichterle labs; (c) mouse breeding is nearly complete. If we do not identify a potent shRNA for PLD1 we will use the CRISPR technology developed by the Wichterle lab to create homozygous Pld1 null ES/iPS lines and produce motor neurons from those. If no effect is observed in the SOD1 models, we will expand the number of sALS lines studied. If still no effect is observed we will discount PLD1 as a target—an appropriate outcome for an R21—and proceed with Specific Aim 2.

Specific Aim 2: Evaluation of Other Candidate Modifier Genes.

There is also strong biological rationale for many of the other genes in Table 1, and for two of them (HIPPI and mitofusin) we already have preliminary data showing that their overexpression in human ES-MNs can trigger neurodegeneration. Using the same approaches as in Aim 1.1, we will assess the effects of genetic modulation of their levels in the full range of cell-autonomous and non-cell-autonomous in vitro models. In cases where full-length cDNAs are not available from the Broad library, we have much experience in development of appropriate viral vectors and, as above, CRISPR will be used wherever problems are encountered with specific shRNAs.

Potential pitfalls: In many cases the expected outcome is an increase in the ALS phenotype at higher levels of expression. However, it will be important to interpret cautiously the effects of overexpression studies. For those genes that show initially positive results we will attempt to ascertain specificity through a series of approaches: (a) moderate overexpression at levels comparable to those in the post mortem motor neurons; (b) overexpression of mutant forms of the gene expected to be inactive; (c) inhibition of the specific downstream pathway expected to be triggered, using small-molecule inhibitors or shRNAs. The latter experiments will also provide the first mechanistic insight into the role of each gene, which will be more fully explored using other funding mechanisms.

Future studies: The proposed experiments are only a first stage in evaluating the therapeutic potential of any of the candidate disease modifiers. If results for PLD1 are positive, we will already have a strong case for target validation in ALS and so it will be important to ask whether PLD1 can be modulated through more clinically-relevant approaches, and at stages after disease onset. This should be possible using the existing inhibitors, once we have established that they effectively inhibit PLD1 in spinal motor neurons over a sufficient period. For the other genes (and for PLD1 if hurdles are encountered with in vivo administration of the inhibitors), our collaboration with the UNC AAV vector core should allow for expeditious in vivo validation of any that show effects in the in vitro models in both ALS and AD mice.

Although illustrative embodiments of the present invention have been described herein, it should be understood that the invention is not limited to those described, and that various other changes or modifications may be made by one skilled in the art without departing from the scope or spirit of the invention.

### OTHER REFERENCES

Boillee, S., Yamanaka, K., Lobsiger, C. S., Copeland, N. G., Jenkins, N. A., Kassiotis, G., Kollias, G., Cleveland, D. W., 2006. Onset and progression in inherited ALS determined by motor neurons and microglia. Science 312, 1389-1392.
Boulting, G. L., Kiskinis, E., Croft, G. F., Amoroso, M. W., Oakley, D. H., Wainger, B. J., Williams, D. J., Kahler, D. J., Yamaki, M., Davidow, L., Rodolfa, C. T., Dimos, J. T., Mikkilineni, S., MacDermott, A. B., Woolf, C. J., Henderson, C. E., Wichterle, H., Eggan, K., 2011. A functionally characterized test set of human induced pluripotent stem cells. Nat Biotechnol 29, 279-286.
Cai, D., Zhong, M., Wang, R., Netzer, W. J., Shields, D., Zheng, H., Sisodia, S. S., Foster, D. A., Gorelick, F. S., Xu, H., Greengard, P., 2006. Phospholipase D1 corrects impaired betaAPP trafficking and neurite outgrowth in familial Alzheimer's disease-linked presenilin-1 mutant neurons. Proc Natl Acad Sci USA 103, 1936-1940.
Chen, H., Chan, D. C., 2005. Emerging functions of mammalian mitochondrial fusion and fission. Hum Mol Genet 14 Spec No. 2, R283-289.
Chen, J., Lu, Y., Xu, J., Huang, Y., Cheng, H., Hu, G., Luo, C., Lou, M., Cao, G., Xie, Y., Ying, K., 2004. Identification and characterization of NBEALI, a novel human neurobeachin-like 1 protein gene from fetal brain, which is up regulated in glioma. Brain Res Mol Brain Res 125, 147-155.
Dall'Armi, C., Devereaux, K., Di Paolo, G., 2013. The role of lipids in the control of autophagy. Current Biol. 23, R33-R45.
Dall'Armi, C., Hurtado-Lorenzo, A., Tian, H., Morel, E., Nezu, A., Chan, R. B., Yu, W. H., Robinson, K. S., Yeku, 0., Small, S. A., Duff, K., Frohman, M. A., Wenk, M. R., Yamamoto, A., Di Paolo, G., 2010. The phospholipase D1 pathway modulates macroautophagy. Nat Commun 1, 142.
Datta, M., Bhattacharyya, N. P., 2011. Regulation of RE1 protein silencing transcription factor (REST) expression by HIP1 protein interactor (HIPPI). J Biol Chem 286, 33759-33769.
Di Giorgio, F. P., Boulting, G. L., Bobrowicz, S., Eggan, K. C., 2008. Human embryonic stem cell-derived motor neurons are sensitive to the toxic effect of glial cells carrying an ALS-causing mutation. Cell Stem Cell 3, 637-648.
Dimos, J. T., Rodolfa, K. T., Niakan, K. K., Weisenthal, L. M., Mitsumoto, H., Chung, W., Croft, G. F., Saphier, G., Leibel, R., Goland, R., Wichterle, H., Henderson, C. E., Eggan, K., 2008. Induced pluripotent stem cells generated from patients with ALS can be differentiated into motor neurons. Science 321, 1218-1221.
Fecto, F., Yan, J., Vemula, S. P., Liu, E., Yang, Y., Chen, W., Zheng, J. G., Shi, Y., Siddique, N., Arrat, H., Donkervoort, S., Ajroud-Driss, S., Sufit, R. L., Heller, S. L., Deng, H. X., Siddique, T., 2011. SQSTMI mutations in familial and sporadic amyotrophic lateral sclerosis. Arch Neurol 68, 1440-1446.
Ferrera, L., Caponnetto, C., Marini, V., Rizzi, D., Bordo, D., Penco, S., Amoroso, A., Origone, P., Game, C., 2003. An Italian dominant FALS Leu144Phe SOD1 mutation: genotype-phenotype correlation. Amyotroph Lateral Scler Other Motor Neuron Disord 4, 167-170.
Filimonenko, M., Isakson, P., Finley, K. D., Anderson, M., Jeong, H., Melia, T. J., Bartlett, B. J., Myers, K. M., Birkeland, H. C., Lamark, T., Krainc, D., Brech, A., Stenmark, H., Simonsen, A., Yamamoto, A., 2010. The selective macroautophagic degradation of aggregated proteins requires the PI3P-binding protein Alfy. Mol Cell 38, 265-279.
Gervais, F. G., Singaraja, R., Xanthoudakis, S., Gutekunst, C. A., Leavitt, B. R., Metzler, M., Hackam, A. S., Tam, J., Vaillancourt, J. P., Houtzager, V., Rasper, D. M., Roy, S., Hayden, M. R., Nicholson, D. W., 2002. Recruitment and activation of caspase-8 by the Huntingtin-interacting protein Hip-1 and a novel partner Hippi. Nat Cell Biol 4, 95-105.
Haidet-Phillips, A. M., Hester, M. E., Miranda, C. J., Meyer, K., Braun, L., Frakes, A., Song, S., Likhite, S., Murtha, M. J., Foust, K. D., Rao, M., Eagle, A., Kammesheidt, A., Christensen, A., Mendell, J. R., Burghes, A. H., Kaspar, B. K., 2011. Astrocytes from familial and sporadic ALS patients are toxic to motor neurons. Nat Biotechnol 29, 824-828.
Kanning, K. C., Kaplan, A., Henderson, C. E., 2010. Motor neuron diversity in development and disease. Annu Rev Neurosci 33, 409-440.
Kaplan, A., Spiller, K. J., Towne, C., Kanning, K. C., Choe, G. T., Geber, A., Akay, T., Aebischer, P., Henderson, C. E., 2014. Neuronal matrix metalloproteinase-9 is a determinant of selective neurodegeneration. Neuron 81, 333-348.
Kiskinis, E., Sandoe, J., Williams, L. A., Boulting, G. L., Moccia, R., Wainger, B. J., Han, S., Peng, T., Thams, S., Mikkilineni, S., Mellin, C., Merkle, F. T., Davis-Dusenbery, B. N., Ziller, M., Oakley, D., Ichida, J., Di Costanzo, S., Atwater, N., Maeder, M. L., Goodwin, M. J., Nemesh, J., Handsaker, R. E., Paull, D., Noggle, S., McCarroll, S. A., Joung, J. K., Woolf, C. J., Brown, R. H., Eggan, K., 2014. Pathways Disrupted in Human ALS Motor Neurons Identified through Genetic Correction of Mutant SOD1. Cell Stem Cell.
Koshiba, T., Detmer, S. A., Kaiser, J. T., Chen, H., McCaffery, J. M., Chan, D. C., 2004. Structural basis of mitochondrial tethering by mitofusin complexes. Science 305, 858-862.
Kuba, H., Oichi, Y., Ohmori, H., 2010. Presynaptic activity regulates Na(+) channel distribution at the axon initial segment. Nature 465, 1075-1078.
Manjaly, Z. R., Scott, K. M., Abhinav, K., Wijesekera, L., Ganesalingam, J., Goldstein, L. H., Janssen, A., Dougherty, A., Willey, E., Stanton, B. R., Turner, M. R., Ampong, M. A., Sakel, M., Orrell, R. W., Howard, R., Shaw, C. E., Leigh, P. N., Al-Chalabi, A., 2010. The sex ratio in amyotrophic lateral sclerosis: A population based study. Amyotroph Lateral Scler 11, 439-442.
Mask G., Ros, S., Gemma, A., Bonfigli, L., Carraro, N., Cazzato, G., Rolfo, M., Zanconati, F., Sepcic, J., Jurjevic, A., Pirulli, D., Boniotto, M., Zezlina, S., Crovella, S., Amoroso, A., 2001. ALS with variable phenotypes in a six-generation family caused by leu144phe mutation in the SOD1 gene. J Neurol Sci 191, 11-18.
Medrihan, L., Rohlmann, A., Fairless, R., Andrae, J., D6ring, M., Missler, M., Zhang, W., Kilimann, M. W., 2009. Neurobeachin, a protein implicated in membrane protein traffic and autism, is required for the formation and functioning of central synapses. J Physiol 587, 5095-5106.
Nagai, M., Re, D. B., Nagata, T., Chalazonitis, A., Jessell, T. M., Wichterle, H., Przedborski, S., 2007. Astrocytes expressing ALS-linked mutated SOD1 release factors selectively toxic to motor neurons. Nat Neurosci 10, 615-622.
Niesmann, K., Breuer, D., Brockhaus, J., Born, G., Wolff, I., Reissner, C., Kilimann, M. W., Rohlmann, A., Missler, M., 2011. Dendritic spine formation and synaptic function require neurobeachin. Nat Commun 2, 557.
Point Du Jour, K., Chan, R., Dall'Armi, C., Lasiecka, Z., Oliveira, T., Shui, G., Wenk, M., Di Paolo, G., 2014. Phospholipase D1-derived phosphatidic acid modulates the metabolism of amyloid precursor protein through gamma-secretase in a mouse model of Alzheimer's disease. submitted.
Rabin, S. J., Kim, J. M., Baughn, M., Libby, R. T., Kim, Y. J., Fan, Y., La Spada, A., Stone, B., Ravits, J., 2010. Sporadic ALS has compartment-specific aberrant exon splicing and altered cell-matrix adhesion biology. Hum Mol Genet 19, 313-328.
Re, D. B., Le Verche, V., Yu, C., Amoroso, M. W., Politi, K. A., Phani, S., Ikiz, B., Hoffmann, L., Koolen, M., Nagata, T., Papadimitriou, D., Nagy, P., Mitsumoto, H., Kariya, S., Wichterle, H., Henderson, C. E., Przedborski, S., 2014. Necroptosis drives motor neuron death in models of both sporadic and familial ALS. Neuron 81, 1001-1008.
Roybon, L., Lamas, N. J., Garcia-Diaz, A., Yang, E. J., Sattler, R., Jackson-Lewis, V., Kim, Y. A., Kachel, C. A., Rothstein, J. D., Przedborski, S., Wichterle, H., Henderson, C. E., 2013. Human Stem Cell-Derived Spinal Cord Astrocytes with Defined Mature or Reactive Phenotypes. Cell Rep.
Saxena, S., Caroni, P., 2011. Selective neuronal vulnerability in neurodegenerative diseases: from stressor thresholds to degeneration. Neuron 71, 35-48.
Schon, E. A., Przedborski, S., 2011. Mitochondria: the next (neurode)generation. Neuron 70, 1033-1053.
Selvy, P. E., Lavieri, R. R., Lindsley, C. W., Brown, H. A., 2011. Phospholipase D: enzymology, functionality, and chemical modulation. Chem Rev 111, 6064-6119.
Su, Y., Balice-Gordon, R. J., Hess, D. M., Landsman, D. S., Minarcik, J., Golden, J., Hurwitz, I., Liebhaber, S. A., Cooke, N. E., 2004. Neurobeachin is essential for neuromuscular synaptic transmission. J Neurosci 24, 3627-3636.
Turner, B. J., Talbot, K., 2008. Transgenics, toxicity and therapeutics in rodent models of mutant SOD1-mediated familial ALS. Prog Neurobiol 85, 94-134.
Valdez, G., Tapia, J. C., Lichtman, J. W., Fox, M. A., Sanes, J. R., 2012. Shared resistance to aging and ALS in neuromuscular junctions of specific muscles. PLoS One 7, e34640.
Wainger, B. J., Kiskinis, E., Mellin, C., Wiskow, 0., Han, S. S., Sandoe, J., Perez, N. P., Williams, L. A., Lee, S., Boulting, G., Berry, J. D., Brown, R. H., Cudkowicz, M. E., Bean, B. P., Eggan, K., Woolf, C. J., 2014. Intrinsic membrane hyperexcitability of amyotrophic lateral sclerosis patient-derived motor neurons. Cell Rep 7, 1-11.
Wang, X., Herberg, F. W., Laue, M. M., Wullner, C., Hu, B., Petrasch-Parwez, E., Kilimann, M. W., 2000. Neurobeachin: A protein kinase A-anchoring, beige/Chediak-higashi protein homolog implicated in neuronal membrane traffic. J Neurosci 20, 8551-8565.
Yamanaka, K., Chun, S. J., Boillee, S., Fujimori-Tonou, N., Yamashita, H., Gutmann, D. H., Takahashi, R., Misawa, H., Cleveland, D. W., 2008. Astrocytes as determinants of disease progression in inherited amyotrophic lateral sclerosis. Nat Neurosci 11, 251-253.
Yoon, M. S., Yon, C., Park, S. Y., Oh, D. Y., Han, A. H., Kim, D. S., Han, J. S., 2005. Role of phospholipase D1 in neurite outgrowth of neural stem cells. Biochem Biophys Res Commun 329, 804-811.
Zorzano, A., Liesa, M., Sebastian, D., Segales, J., Palacin, M., 2010. Mitochondrial fusion proteins: dual regulators of morphology and metabolism. Semin Cell Dev Biol 21, 566-574.

1. A method for treating or ameliorating an effect of amyotrophic lateral sclerosis (ALS) comprising administering to a subject in need thereof a modulator of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPO; zinc finger RNA-binding motif sennetarginine rich 2 U2AF35-related protein (ZRSR2), and combinations thereof, in an amount effective to treat or ameliorate an effect of amyotrophic lateral sclerosis (ALS).

2. A method for preventing or slowing motor neuron disease in a subject comprising administering to a subject in need thereof a modulator of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPO; zinc finger RNA-binding motif sennetarginine rich 2 U2AF35-related protein (ZRSR2), and combinations thereof in an amount effective to prevent or slow motor neuron disease.

3. A pharmaceutical composition for treating or ameliorating an effect of amyotrophic lateral sclerosis (ALS) in a subject in need thereof, the pharmaceutical composition comprising a pharmaceutically acceptable carrier or diluent and an amount of a modulator of a gene selected from the group consisting of Phospholipase D1 (PLD1); Polymerise (DNA-directed), delta 3, accessory subunit (POLD3); Aminoacyl tRNA synthetase complex-interacting multifunctional protein 1 (AIMP1); Sterol O-acyltransferase 1 (SOAT1); LSMB: N(alpha)-acetyltransferase 38, NatC auxiliary subunit (NAA38); Lysine specific demethyrase 58 (KDM5B); Mitofusin 1 (MFN1); MOP-1 (MOP-1); Solute carrier family 30 (zinc transporter), member 7 (SLC30A7); ALS2CR16: neurobeachin-like 1 (NBEAL1); Solute carrier family 4, sodium bicarbonate cotransporter, member 7 (SLC4A7); Protein geranylgeranyltransferase type I, beta subunit (PGGT1B); Taste receptor, type 2, member 4 (TAS2R4); Histone cluster 1, H2bc (HIST1H2BC); Intraflegellar transport 57 homolog (IFT57) (HIPPI); zinc finger RNA-binding motif sennetarginine rich 2 U2AF35-related protein (ZRSR2), and combinations thereof, which amount is effective to treat or ameliorate an effect of amyotrophic lateral sclerosis (ALS) in the subject.

4. The method or composition of claims 1-3, wherein the gene is selected from the group consisting of Phospholipase D1 (PLD1); Intraflegellar transport 57 homolog (IFT57) (HIPPI); ALS2CR16: neurobeachin-like 1 (NBEAL1); Mitofusin 1 (MFN1); Protein geranylgeranyltransferase type I, beta subunit (PGGT1B), and combinations thereof.

5. The method or composition of claims 1-3, wherein the gene is Phospholipase D1 (PLD1).

6. The method or composition of claims 1-3, wherein the modulator is an activator of the gene.

7. The method or composition of claims 1-3, wherein the modulator is an inhibitor of the gene.

8. The method or composition of claims 1-3, wherein the modulator is a small molecule.
